(19) United States
(12) Patent Application Publication
(10) Pub. No.: US 2010/0166836 A1
(43) Pub. Date: Jul. 1, 2010

(54) Transdermal Therapeutic System for Volatile and/or Thermo-Labile Substances

(76) Inventors: Tobias Jung, Efringen-Kirchen (DE); Michael Horstmann, Neuwied (DE); Horst Dzekan, Meinborn (DE)

Correspondence Address: FROMMER LAWRENCE & HAUG, 745 FIFTH AVENUE- 10TH FL., NEW YORK, NY, 10151, US

(21) Appl. No.: 12/160,641

(22) PCT Filed: Dec. 16, 2006
(86) PCT No.: PCT/EP06/12149

§ 371 Date: Jul. 11, 2008

(30) Foreign Application Priority Data
Jan. 12, 2006 (DE) 10 2006 001 536.3
Jun. 3, 2006 (DE) 10 2006 026 060.0

(51) Int. Cl. A61K 31/4439; A61K 9/70; A61P 25/34; A61F 13/02

(52) U.S. Cl. 424/448; 424/449; 514/343

(57) ABSTRACT

The present invention relates to a transdermal therapeutic system comprising at least one readily volatile and/or thermolabile active ingredient and/or auxiliary, which can be produced by laminating at least three components onto one another, namely a polymeric matrix layer, a likewise polymeric acceptor layer that absorbs the active ingredient and/or auxiliary at an increased rate, and a donor layer comprising, at the point of production, the volatile and/or thermolabile active ingredient and/or auxiliary. The system according to the invention is characterized in that, during or shortly after the lamination process, the donor layer combines with the acceptor layer as a result of migration of the readily volatile and/or thermolabile substances.

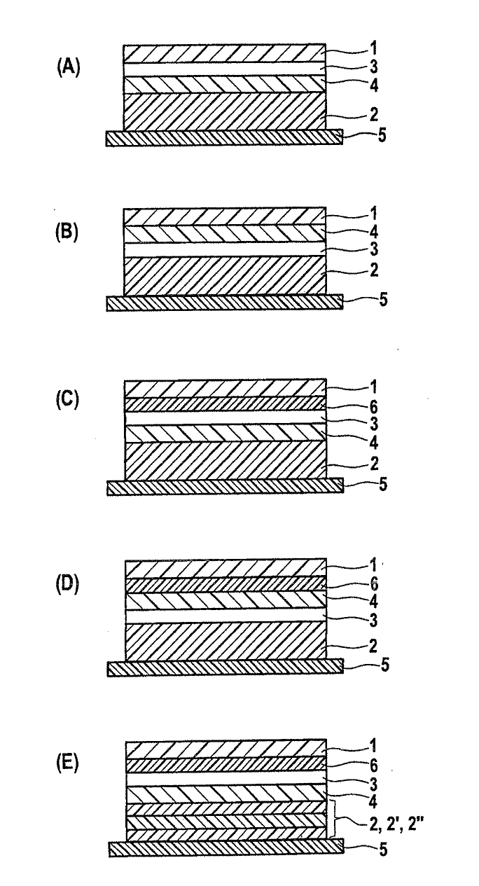

Transdermal therapeutic systems (TTS) are administration forms which are applied to the skin and are designed to make a drug available systemically following transdermal absorption. TTS are able to increase the therapeutic value of drug administration by ensuring constant delivery of the active into the blood compartment over a prolonged time period. The advantages of this continuous delivery of active are, primarily, the extended intervals of application, leading to improved patient compliance, and the pharmacokinetically optimized plasma concentration/time profile, which ensures a longer duration of action with fewer side effects. Further advantages occasioned by the transdermal application route are improved gastrointestinal compatibility and improved bioavailability as a result of avoidance of the first-pass effect.

On the basis of all of these advantages, TTS have been known for some years. Systems of this kind have been introduced into therapy for actives including, for example, estradiol, norethisterone acetate, nicotine, fentanyl, tulobuterol, ethinylestradiol/norelgestromine, buprenorphine or nitroglycerine, and a series of further actives. Their construction is generally thin and layered, thereby producing, with the aid of the side directly facing the skin (adhesive layer), an at least temporarily adhesive bond to the skin, via which the active is delivered. TTS are typically composed, in accordance with the prior art, of a drug-impervious backing layer, a drug-containing reservoir layer or matrix layer, and a pressure-sensitive adhesive layer for attachment to the skin, this layer possibly being identical with the drug-containing reservoir or matrix layer, and a drug-impervious protective layer (release liner) intended for removal prior to application.

To improve the permeation of active through the skin use is made, in addition to polymers, resins, and other pharmaceutical auxiliaries, of system components which are liquid at room temperature and which serve in part to adjust the bond strength, to improve diffusion within the transdermal therapeutic system or else to improve the permeation of active through the skin.

Not only actives but also primarily liquid auxiliaries may have the property, which is disruptive during production, of volatility and/or thermolability under operating conditions. One possible consequence of this is the occurrence of significant losses during the production of transdermal systems, which typically consists in the mixing of the starting materials in a suitable organic solvent, subsequent coating in a thin layer on a base film, and subsequent, usually continuous, drying at elevated temperature.

These losses, more particularly those from the last stage of production, that of drying, can lead to instances of misdosing and/or performance deficiencies on the part of the TTS produced, which therefore restrict or render impossible a regular production and hence a therapeutic application, more particularly in humans.

Without making any claim to an exhaustive recitation, mention may be made, by way of example, as volatile and/or thermolabile auxiliaries, of the following:

2-pyrrolidone, benzyl alcohol, butanol, butanediol and other short-chain alcohols, cineol, diethylene glycol, diethylene glycol monoethyl ether, diisopropyl adipate, dodecanol, dimethyldecyl phosphoxide, dimethylisosorbide, dimethyllauroylamide, polydimethylsiloxane, dimethyl sulfoxide, dodecyl sulfoxide, acetic acid, ethyl acetate and other volatile aliphatic and aromatic esters (which are mixture constituents of many essential oils), ethylene glycol, ethylene glycol monolaurate and other esters and ethers of ethylene glycol or propylene glycol, 2-octyldodecanol, glycerol, glycerol monooleate, glycerol monostearate, hydrogenated castor oil, isopropyl myristate, isopropyl palmitate, menthol or other volatile terpene derivatives (which are mixture constituents of many natural essential oils), methyl benzoate, methyl octyl sulfoxide, mono- or diethylacetamide, N,N-diethyl-m-toluamide, N-methylpyrrolidone, octan-1-ol and other volatile medium-chain alcohols, octanoic acid and other medium-chain aliphatic carboxylic acids, oleyl alcohol, propanediol, olive oil, oleic acid, oleyl oleate, phenylethanol, propylene glycol, ricinoleic acid, Transcutol®, triacetin, and also mixtures of such compounds, such as oleic acid/propylene glycol or limonene/dimethylisosorbide, for example.

Preferred volatile and/or thermolabile auxiliaries include the following: cineol, diethylene glycol, dodecanol, ethylene glycol, propylene glycol, menthol, terpene derivative, N,N-diethyl-m-toluamide, propanediol, and Transcutol®.

The highly-volatile and/or thermolabile actives include, for example, nicotine, nitroglycerine, bupropion, salicylic acid, mecamylamine, selegiline, scopolamine, venlafaxine, oxybutynin, benzatropine, fenfluramine, tulobuterol, fentanyl, sufentanil, capsaicin, methyl salicylate, cyclopentamine, ephedrine, without this listing being exhaustive. Mixtures of these substances, independently of the active or auxiliary nature, may be used in the same way and may be particularly advantageous.

Beyond the above property of volatility, the possibility exists, when dry or wet heat is used during the drying of the transdermal therapeutic systems, of premature breakdown of actives or auxiliaries as a result of exposure to heat, oxygen, and humidity in conjunction with a high level of convection of the drying vehicles (gases, more particularly air).

For these reasons, conventional methods of producing transdermal therapeutic systems are considered entirely problematic and frequently cannot be employed directly (see, for example, “Dermatological Formulation and Transdermal Systems”, Kenneth A. Walters and Keith R. Brain, in Dermatological and Transdermal Formulations, New York 2002, Marcel Dekker, pages 319-399, production more particularly at page 343, bottom).

There has in the past been no lack of attempts to develop production processes for avoiding said losses of material through volatility and/or thermolability. Mention may accordingly be made here, by way of example, of the solutions proposed in DE 36 29 304, U.S. Pat. No. 4,915,950 and, more particularly, U.S. Pat. No. 5,902,601. In the latter a preparation of the active or auxiliary, that has been made by addition of Polymeisko, is coated exactly onto a substrate and is laminated with a tacky, active-free adhesive layer in such a way as to form, after migration of the active or auxiliary, a matrix which overall is solidified. The disadvantage of this prior-art process, however, as also conceded by example 2 of U.S. Pat. No. 5,902,601, is the relatively slow “equilibration time” (given there as 60 min) required until the resulting product becomes a system possessing overall shear resistance. Since, accordingly, each individual site in the laminate resulting from industrial production must not be subjected to mechanical loading in the ongoing production operation, such an operation can hardly be implemented industrially with low-diffusibility polymers, such as polyisobutylene, styrene-isoprene copolymers, and even with certain acrylate polymers.

DE 43 32 094 C2 describes a solventlessly producible active-substance patch which allows the virtually loss-free incorporation of auxiliaries or actives which are volatile at the typical processing temperature. This is achieved by laminating a first matrix layer, which during production constitutes a spreadable, molecular-disperse solution of the matrix base material in the highly-volatile active or auxiliary as the exclusive solvent, onto a separately produced assembly comprising one or more further matrix layers. As a result of subsequent migration of the high-volatility active or auxiliary into the bordering matrix, the initially spreadable, highly viscous consistency of the first matrix layer is lost, and the system as a whole becomes soft and tacky, but dimensionally stable or shear-stable, in the way which is necessary for use as an active-substance patch. The duration of this procedure (“aging process”) is dependent, alongside other physical parameters, on the diffusion properties of all the ingredients and also on the overall geometry; it amounts to a few minutes up to several hours, but in certain cases may even amount to several days up to a week. It is apparent that one possible consequence of the different long “aging process” is that a TTS which is used a short time after production has not yet attained the required dimensional stability. Moreover, the fact that the first matrix layer, which after the “aging time” is now free of active or auxiliary, remains in the completed TTS entails additional thickening of the system.

The known prior art further reveals that, in the case of the production of a transdermal therapeutic system with highly-volatile and/or thermolabile ingredients, not just any combination of matrix base polymer/ingredient is possible, but that, in contrast, the possibilities for combination are very limited. Thus, for example, a transdermal therapeutic system comprising nicotine as a (volatile) active cannot be produced on the basis of a polyisobutylene matrix, since the nicotine, when applied to the matrix, would remain on it in the form of a floating liquid layer.

The object of the present invention, therefore, is to provide a dimensionally stable transdermal therapeutic system comprising volatile and/or thermolabile actives and/or auxiliaries that avoids the disadvantages of the comparable systems known from the prior art, and more particularly also extends the possibilities for combination of matrix base polymer with volatile and/or thermolabile ingredients.

This object is achieved by means of a transdermal therapeutic system in accordance with claim 1.

The laminating of the system components to one another can be performed in any order. Hence the skin-side polymeric matrix layer (2), which may also be multilayer (2′,2″), can be laminated first to the acceptor layer (4) and then to the donor layer (3). An alternative option is to proceed in the opposite order: for example, a skin-side layer (2) may be followed directly also by layer (3), then by layer (4), and then, for example, finally, by an active-impervious backing layer (1). The surprising success according to the invention is a product of the property of layer (4) to rapidly take on the volatile and/or thermolabile component from layer (3) and, in so doing, to first make the system shear-resistant, before, after a number of hours of further migration, a large part of the volatile and/or thermolabile component has diffused into the layer (3) containing the lower-diffusability polymer. The further construction of the system according to the sensation, and its converting, are accomplished with the typical components and techniques, known to the skilled worker, of lamination, of winding, of singularization, diecutting, etc.

Typically at least one additional, generally active-impervious backing layer (backing 1) is added, and further, where the matrix (2) does not already have adhesive properties, an adhesive layer is applied toward the skin, and also, for the keeping of the transdermal therapeutic system, a redetachable protective layer (release liner 5), which is removed prior to the use of the system. Additionally it would be possible, for a better bond of the donor layer or, depending on construction, of the acceptor layer to the backing layer, for an adhesive anchor layer (6) to be present. In spite of their multilayered nature, the systems according to the invention do not have any disadvantages from a performance standpoint, since, given an appropriate choice of layer thicknesses, the systems go on with the same thickness, or even more thinly, than prior-art systems. Advantageous as compared with the prior art is the extremely accelerated processibility which is provided in conjunction with protection from temperature-induced volatility or instability of the components, and which results in such systems being producible on the industrial scale.

Furthermore, the art of formulation is opened up to polymers, more particularly adhesive polymers, whose slow permeation characteristics for actives and auxiliaries had hitherto made them unsuitable, from the production standpoint, for migration methods. Such polymers include more particularly the following groups of polymer very typical in transdermal systems: styrene-isoprene copolymers, polyisobutylenes, acrylate copolymers, butyl rubber, polybutylenes, and a number of other, comparable polymers with significantly lower absorption potential.

The nature of the volatile and/or thermolabile components and the candidates under consideration have already been described in the introductory section. The following are possible base polymers for the matrix layer (2): polyisobutylene, polybutylene, styrene-isoprene, styrene copolymer, styrene-butadiene-styrene block copolymer. In this context it is possible to utilize further components such as resins, oils, fillers, and other pharmaceutically typical modifiers.

As base polymers suitable for the matrix layer(s), preference is given to polyisobutylene, polybutylene, and butyl rubber, more particularly polyisobutylene and polybutylene.

The layer can be produced by conventional techniques of dissolving, mixing, coating, and temperature-protected drying, since it does not yet contain the volatile and/or thermolabile component. The same applies to the acceptor layer (4), which, however, is composed not of low-diffusibility components but instead of polymers having significantly accelerated absorption characteristics for actives and auxiliaries. The layer may be given a tacky or nontacky formulation. Here, therefore, exemplary base polymers can be made up with water-solubility and with organic polymer solubility. Mention may be made, by way of example, of substances such as polyvinyl alcohol, polyvinyl acetate, silicone adhesives and silicone rubbers, and acrylate and methyl acrylate copolymers.

It is entirely possible, however, for lower-diffusibility polymers and adjuvants to be used, provided that accelerated active-absorption characteristics still always result relative to layer (2). In one particular embodiment this can also be accomplished by imbedding particles of strongly swelling substances (PVP, polyvinyl alcohol) in a base matrix comprising low-diffusibility polymers.

### EXAMPLES

### Example 1

a) A solution comprising a mixture of low and high molecular mass polyisobutylene, polybutylene, and an aliphatic hydrocarbon is applied to an intermediate siliconized 36 μm PET film (anchor layer (6)) in such a way that drying results in a coatweight of approximately 110 g/m². After drying, this coat of adhesive is lined with a transparent 23 μm PET film (1).
b) In a second workstep, the same polymer solution from (a) is applied to a siliconized 100 μm PET film (5) in such a way that drying results in a coat of adhesive of approximately 110 g/m² (2). This coat of adhesive is lined with the siliconized side of a 36 μm PET film.
c) Subsequently a solution of poly(butyl methacrylate) and ethyl acetate is coated onto an intermediate unsiliconized 36 μm PET film in such a way that drying results in a coatweight of approximately 90 g/m² (4). Immediately after drying, the film of poly(butyl methacrylate, methylene acrylate) is laminated onto the coat of adhesive from (b) (the 36 μm PET film is peeled off beforehand).
d) Thereafter a solution of nicotine and poly(butyl methacrylate, methylene acrylate) is coated onto the poly(butyl methacrylate, methylene acrylate) film in such a way as to result in a coatweight of approximately 60 g/m² (3). Finally, following removal of the 36 μm PET film, the anchor layer from (a) is laminated onto the assembly comprising 100 μm PET film (5), matrix (2), poly(butyl methacrylate, methylene acrylate) (4), and nicotine/poly(butyl methacrylate, methylene acrylate) (3). The result is the completed nicotine laminate. The transdermal therapeutic systems of versions 1-4 are produced correspondingly.

| Example 1 | | Version 1 | | Version 2 | | Version 3 | | Version 4 | |
|---|---|---|---|---|---|---|---|---|---|
| (FIG. C) | | (FIG. A) | | (FIG. B) | | (FIG. D) | | (FIG. E) | |
| 1 | PET film, 23 μm | 1 | PET film, 23 μm | 1 | PET film, 23 μm | 1 | PET film, 23 μm | 1 | PET film, 23 μm |
| | transp. | | transp. | | transp. | | transp. | | transp. |
| 6 | PIB/PB matrix, | 3 | Nic/Plastoid B, | 4 | Plastoid B, approx. | 6 | PIB/PB matrix, | 6 | PIB/PB matrix, |
| | approx. 110 g/m² | | 1.4:1 approx. 60 g/m² | | 90 g/m² | | approx. 110 g/m² | | approx. 110 g/m² |
| 3 | Nic/Plastoid B, | 4 | Plastoid B, approx. | 3 | Nic/Plastoid B, | 4 | Plastoid B, approx. | 3 | Nic/Plastoid B, |
| | approx. 1.4:1 | | 90 g/m² | | 1.4:1 approx. 60 g/m² | | 90 g/m² | | 1.4:1 approx. |
| | approx. 60 g/m² | | | | | | | | 60 g/m² |
| 4 | Plastoid B, | 2 | PIB/PB matrix, | 2 | PIB/PB matrix, | 3 | Nic/Plastoid B, 1.4:1 | 4 | Plastoid B, |
| | approx. 90 g/m² | | approx. 110 g/m² | | approx. 110 g/m² | | approx. 60 g/m² | | approx. 90 g/m² |
| 2 | PIB/PB matrix, | 5 | PET film 100 μm | 5 | PET film 100 μm | 2 | PIB/PB matrix, | 2 | PIB/PB matrix, |
| | approx. 110 g/m² | | transp. | | transp. | | approx. 110 g/m² | | approx. 110 g/m² |
| 5 | PET film 100 μm | | | | | 5 | PET film 100 μm | (2′2″) | GSM 3083, approx. |
| | transp. | | | | | | transp. | | 40 g/m² |
| | | | | | | | | 5 | PET film 100 μm |
| | | | | | | | | | transp. |

1 = Backing
2 = Matrix
2′2″ = Optional multilayer matrix
3 = Donor
4 = Acceptor
5 = Release Liner
6 = Optional anchor layer

1-9. (canceled)

10. A transdermal therapeutic system comprising at least one highly-volatile and/or thermolabile active and/or auxiliary, composed of a backing layer which is substantially impervious to the active and the auxiliary, at least two polymeric matrix layers following the backing layer, of which at least one contains the active and/or the auxiliary, and of a subsequent redetachable protective layer, characterized in that said transdermal therapeutic system is producible by laminating to one another the following, mutually bordering layers:
a) a substantially active-impervious backing layer,
b) if desired, a matrix layer serving as anchor layer,
c) a polymeric matrix layer which serves as donor layer and which at the time of production of the transdermal therapeutic system contains the highly-volatile and/or thermolabile actives and, where appropriate, auxiliaries,
d) a polymeric matrix layer which serves as an acceptor layer,
e) a further skin-side polymeric matrix layer,
f) a redetachable protective layer,
the acceptor layer possessing the property of accelerated absorption of said active and/or auxiliary, and the donor layer uniting with the acceptor layer, as a result of migration of the highly-volatile and/or thermolabile actives and/or auxiliaries into said acceptor layer, during lamination or a short time after lamination.

11. The transdermal therapeutic system of claim 10, characterized in that the highly-volatile and/or thermolabile active is a pharmaceutical active.

12. The transdermal therapeutic system of claim 10, characterized in that the matrix layer which serves as an anchor layer is bonded pressure-sensitive adhesively to the backing layer.

13. The transdermal therapeutic system of claim 10, characterized in that polyisobutylene, polybutylene, styrene-isoprene, styrene copolymers and/or styrene-butadiene-styrene block polymers are used as base polymers for matrix layer(s), with the exception of donor layer and acceptor layer.

14. The transdermal therapeutic system of claim 13, characterized in that polyisobutylene and/or polybutylene are used as base polymers.

15. The transdermal therapeutic system of claim 1, characterized in that polyvinyl alcohol, polyvinyl acetate, silicone rubbers, acrylate copolymers or methyl acrylate copolymers are used as the base polymer for donor layer and acceptor layer.

16. The transdermal therapeutic system of claim 1, characterized in that the same base polymer is used for donor layer and acceptor layer.

17. The transdermal therapeutic system of claim 16, characterized in that a neutral copolymer based on butyl methacrylate and methyl methacrylate is used as the base polymer for donor layer and acceptor layer.

18. The transdermal therapeutic system of claim 10, characterized in that the skin-side matrix layer possesses adhesive properties.

19. The transdermal therapeutic system of claim 10, characterized in that the skin-side matrix layer is additionally provided with a layer of pressure-sensitive adhesive.

20. The transdermal therapeutic system of claim 10, characterized in that the high-volatility and/or thermolabile active is selected from the group of actives consisting of nicotine, nitroglycerine, bupropion, salicylic acid, mecamylamine, selegiline, scopolamine, oxybutynin, venlafaxine, benzatropine, fenfluramine, tulobuterol, fentanyl, sufentanil, dapsaicin, methyl salicylate, cyclopentamine, and ephedrine.

21. The transdermal therapeutic system of claim 20, characterized in that the active is nicotine.

22. A process for producing a transdermal therapeutic system of claim 10, characterized in that first of all, by coating and drying, an acceptor layer is produced which is suitable for the volatile and/or thermolabile active and/or auxiliary; the volatile and/or thermolabile active and/or auxiliary, either alone or in a mixture with further auxiliaries, is applied as a donor layer to said acceptor layer; the acceptor layer coated in this way is lined directly with a backing layer or first joined to an anchor layer and then lined with a backing layer, and the laminate thus produced is united with at least one matrix provided on the opposite side with a redetachable layer (release liner).

23. The transdermal therapeutic system of claim 11, characterized in that the matrix layer which serves as an anchor layer is bonded pressure-sensitive adhesively to the backing layer.

24. The transdermal therapeutic system of claim 23, characterized in that polyisobutylene, polybutylene, styrene-isoprene, styrene copolymers and/or styrene-butadiene-styrene block polymers are used as base polymers for matrix layer(s), with the exception of donor layer and acceptor layer.

25. The transdermal therapeutic system of claim 24, characterized in that polyisobutylene and/or polybutylene are used as base polymers.

26. The transdermal therapeutic system of claim 25, characterized in that polyvinyl alcohol, polyvinyl acetate, silicone rubbers, acrylate copolymers or methyl acrylate copolymers are used as the base polymer for donor layer and acceptor layer.

27. The transdermal therapeutic system of claim 26, characterized in that the same base polymer is used for donor layer and acceptor layer.

28. The transdermal therapeutic system of claim 27, characterized in that a neutral copolymer based on butyl methacrylate and methyl methacrylate is used as the base polymer for donor layer and acceptor layer.

29. The transdermal therapeutic system of claim 28, characterized in that the active is nicotine.
